(19) United States
(12) Patent Application Publication
(10) Pub. No.: US 2011/0184207 A1
(43) Pub. Date: Jul. 28, 2011

(54) Method of Fabricating Glycol Monoalkyl Ether Acetate Using Acidic Ionic Liquid Catalyst

(75) Inventors: Jung-Chung Wu, Chiayi City (TW); Ming-Yu Huang, Chiayi City (TW); Jen-Chun Chang, Chiayi City (TW); Jann-Chen Lin, Chiayi City (TW)

(73) Assignee: CPC CORPORATION, Taipei City (TW)

(21) Appl. No.: 12/781,065

(22) Filed: May 17, 2010

(30) Foreign Application Priority Data
Jan. 28, 2010 (TW) 099102384

(51) Int. Cl. C07C 67/24

(52) U.S. Cl. 560/186

(57) ABSTRACT

A new method for fabricating glycol monoalkyl ether acetate (GMAEA) is provided. A Bronsted acidic ionic liquid is used. After some reactions, two layers of materials are formed. A product of GMAEA is obtained at the upper layer. The lower layer is the ionic liquid. Thus, the ionic liquid is reusable for re-fabricating the product. And, furthermore, waste acid is reduced.

### TECHNICAL FIELD OF THE DISCLOSURE

The present disclosure relates to fabricating glycol monoalkyl ether acetate (GMAEA); more particularly, relates to using a Bronsted acidic ionic liquid (IL) as a catalyst for fabricating GMAEA by separating product and catalyst in two layers with catalyst recycled and reused and waste acid reduced.

### DESCRIPTION OF THE RELATED ARTS

GMAEA can be synthesized by the reaction of GMAE and acetic acid (HOAC) with an acid catalyst, where the acetate can be ethylene glycol ethyl ether acetate (EGEEA) or diethylene glycol n-butyl ether acetate (DEGnBEA). This kind of compound is a multi-functional solvent having multiple functional groups and a high boiling point. It has a high solvency to polymer and is widely used in fields of coating, printing, rinsing, etc. Its performance is better than ethylene glycol ether and propylene glycol ether. After the environmental protection agency of USA has announced that ethylene glycol ether and related acetates are harmful to health in 80′, the uses of ethylene glycol ether and related acetates are seriously affected. But, because EGEEA has good functions in many fields, it is still used in Europe and USA, where 80˜90% of ethylene glycol monoethyl ether (EGEE) is still used for producing its acetate in USA. Hence, glycol ether ester compound still plays an important role in the market of oxygen-containing solvents.

Technologies for synthesizing glycol ether acetate (GEA) include direct esterification, transesterification and ring-opening esterification.

Regarding the direct esterification, acetic acid (HOAc) and GMAE are used as feedstock with a catalyst, like sulfuric acid, phosphoric acid or toluene sulfonic acid. Yet, product separation and waste water treatment become big problems. It is because that water will be generated in the reaction; separation of the product from acid catalyst usually needs neutralizing and water washing; and glycol ether (GE) and the product of glycol ether ester may be somewhat dissolved in water. Solid acid like ion exchange resin can be used as acid catalyst in substitute. A Japan company used this kind of catalyst and benzene or cyclopentane as a water-carrying agent to remove water generated in reaction for improving the conversion rate. Another American company used a solid acid of Nafion 811 or Amberlyst NX 1010 as a catalyst. The other kinds of solid acids include a strong-acidic ion-exchange resin revealed in 1997; a pillared clay revealed in 1999; TiO₂/SO₄⁻² revealed in 2001; and montmorillonite revealed in U.S. Pat. No. 6,472,555 B2, 2002. The traditional direct esterification is still the main method for producing ether ester solvent through many kinds of improvement.

Regarding the transesterification, ethyl acetate (EtOAc) and GE are used for reaction with a byproduct of ethanol, where no water azeotropic system is generated; the separation procedure is simplified; no HOAc is used; and equipments are less corroded. For example, Shisso Co. Ltd, Japan, uses toluene sulfonic acid as a catalyst for reaction at 77° C. with a yield of 70%. Some other people use an organic metal salt and aluminum alkoxide or titanium alkoxide as catalysts for reaction at a temperature between 150° C. and 225° C., where a high ether ester yield is obtained. But, the product has to be neutralized for removing the homogeneous acid catalyst and has the same water problem. In addition, an azeotropic system of ethanol and ethyl acetate are formed; thus, operational cost is increased.

Regarding the ring-opening esterification, ethylene oxide and ethyl acetate are used. Ethylene oxide costs much lower than GE compound and no water or ester is generated with byproduct of diethylene glycol ethyl ether acetate (DEGEEA) or triethylene glycol ethyl ether acetate (TEGEEA) obtained at a temperature between 120° C. and 180° C. with a 5 Mpa pressure. Although the cost is low and product is easily separated and product purity is high, main product EGEEA does not have a high selectivity and the byproduct does not have a high value. Hence, the prior arts do not fulfill all users' requests on actual use.

### SUMMARY OF THE DISCLOSURE

The main purpose of the present disclosure is to use a Bronsted acidic IL as a catalyst for fabricating GMAEA by separating product and catalyst in two layers with catalyst recycled and reused and waste acid reduced.

To achieve the above purpose, the present disclosure is a method of fabricating GMAEA using acidic IL catalyst, comprising steps of: (a) reacting an organic nitride compound with alkyl sultone to obtain a zwitterion; and, after purifying and drying the zwitterion, processing a reaction with a strong acid having a sulfuric group (—SO₄H) or a sulfonic group (—SO₃H) to obtain a viscous water-tolerable Bronsted acidic IL; (b) adding a hot solution of GMAE and HOAc into the IL to process an acetylation of the GMAE; and (c) after removing CH₃OH and H₂O under vacuum, staying still at a high temperature to obtain two layers of materials, where a lighter layer of the layers is a product and a heavier layer of the layers is the IL. Accordingly, a novel method of fabricating GMAEA using acidic IL catalyst is obtained.

### BRIEF DESCRIPTIONS OF THE DRAWINGS

The present disclosure will be better understood from the following detailed description of the preferred embodiment according to the present disclosure, taken in conjunction with the accompanying drawings, in which

FIG. 1 is the flow view showing the preferred embodiment according to the present disclosure;

FIG. 2 is the view showing the conversion rates for different temperatures;

FIG. 3 is the view showing the conversion rates for different reaction periods of time;

FIG. 4 is the view showing the conversion rates for different mole ratios of reactors;

FIG. 5 is the view showing the conversion rates for different amounts of IL;

FIG. 6 is the view showing the conversion rates for different ILs;

FIG. 7 is the view showing the conversion rates for the ILs containing different amounts of water;

FIG. 8 is the view showing the conversion rates with the water-carrying agent used;

FIG. 9 is the view showing the conversion rates for different diethylene glycol monoalkyl ethers;

FIG. 10 is the view showing the conversion rates for different glycol n-butyl ethers;

FIG. 11 is the view showing the conversion rates for different reused acidic ILs; and

FIG. 12 is the view showing the equipments of the preferred embodiment.

### DESCRIPTION OF THE PREFERRED EMBODIMENT

The following description of the preferred embodiment is provided to understand the features and the structures of the present disclosure.

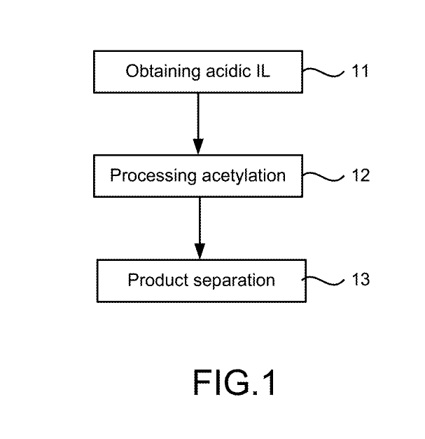

Please refer to FIG. 1, which is a flow view showing a preferred embodiment according to the present disclosure. As shown in the figure, the present disclosure is a method of fabricating glycol monoalkyl ether acetate (GMAEA) using acidic ionic liquid (IL) catalyst. The present disclosure uses a Bronsted acidic IL as a catalyst for fabricating GMAEA through acetylation, comprising the following steps:

(a) Obtaining acidic IL 11: Firstly, an organic nitride compound, including imidazole, pyridine and trialkyl amine, is reacted with an alkyl sultone, like 1,3-propyl or 1,4-butyl sultone, to obtain a white solid of zwitterion through being purified and dried with ether. For example, pyridine can be used to obtain n-propane sulfonic acid pyridinium (PSPy or pyridinium propyl sulfobetaine, PPS). Then, the zwitterion is reacted with a Bronsted strong acid, like a sulfuric acid (H₂SO₄, SA) or a sulfonic acid (R—SO₃H), where the sulfonic acid is fluoro sulfonic acid (FSO₃H, FSA), trifluoro methane sulfonic acid (CF₃SO₃H, TFMSA) or p-toluene sulfonic acid (p-CH₃—C₆H₄—SO₃H, P-TSA). Then, the mixture is stirred under 80° C. for 4 hours to obtain a viscous water-tolerable acidic IL—a Bronsted acidic IL. Therein, a mole ratio of the strong acid to the zwitterion (SA/Zw) is between 1.0 and 1.5—a value between 1.0 and 1.2 is preferred.

(b) Processing acetylation 12: A solution of glycol ether (GE) and acetic acid (HOAc) are added to the IL for acetylation, where a mole ratio of the IL to the glycol ether (IL/GE) is between 0.02 and 1.0; a mole ratio of HOAc to the GE (HOAc/GE) is between 1.0 and 20; the acetylation is processed at a temperature between 25° C. and 120° C. for a period between 0.1 and 5 hours; and a high-temperature reaction is processed in a reflux system or an enclosed reacting system.

(c) Separating product 13: After recycling HOAc and removing water through vacuuming, two layers of materials are formed by staying still. The upper layer is a product and the lower layer is the IL. Then, the product is easily taken out and the IL can be reused for next acetylation.

In step (b), a water-carrying agent of benzene or toluene can be added to improve a conversion rate of the acetylation.

In step (b), the GMAE and HOAc are added into the IL, where the GMAE is ethylene glycol monoalkyl ether, diethylene glycol monoalkyl ether (DEGMAE), propylene glycol monoalkyl ether, di(propylene glycol) monoalkyl ether, etc. For example, it can be ethylene glycol n-butyl ether (EGnBE), diethylene glycol methyl ether (DEGME), diethylene glycol ethyl ether (DEGEE), diethylene glycol n-butyl ether (DEGnBE), diethylene glycol n-hexyl ether (DEGnHE), triethylene glycol n-butyl ether (TEGnBE), propylene glycol n-butyl ether (PGnBE) or dipropylene glycol n-butyl ether (DPGnBE).

Through the above steps, the product is separated from the acid catalyst that the catalyst can be recycled to be reused. Hence, the present disclosure is a green technology.

The present disclosure uses a Bronsted acidic IL as a catalyst to effectively process acetylation of GMAE for producing ether-ester polar solvent having a high boiling point, where the GMAE and HOAc are used in the acetylation and HOAc has an over-amount for a higher conversion rate. The reaction equation of the acetylation of the GMAE is as follows:

HO—[CH₂CHR¹O]ₘ—R²+CH₃COOH→CH₃COO—[CH₂CHR¹O]ₘ—R²+H₂O

Therein, R¹ is H or CH₃; m is a value between 1 and 3; R² is CₙH₂ₙ₊₁; and n is a value between 1 and 9.

The present disclosure uses a Bronsted acidic IL for acetylation to obtain an ether-ester product through a leveling process, where the IL is reusable for next acetylation after removing HOAc and water.

The acidic IL used in the present disclosure is obtained by reacting sulfonic-containing zwitterion with SA, TFMSA, FSA or P-TSA, where HSO₄⁻, CF₃SO₃⁻, FSO₃⁻ or p-CH₃—C₆H₄—SO₃⁻ are conjugated anions. The zwitterion is used as a precursor of the acidic IL; and is obtained by reacting an organic nitride compound with alkyl sultone, whose structure is as follows:

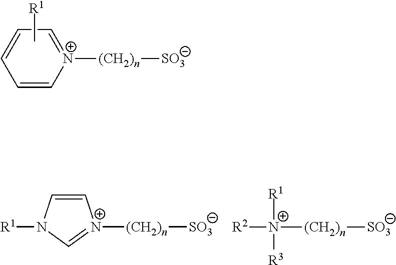

Therein, alkyl (R¹, R² and R³) in the organic nitride compounds, including alkylimidazole, alkylpyridine and alkyl amine, has a chemical formula of CₘH₂ₘ₊₁; m is a value between 1 and 18; alkyl in alkyl sultone is has a chemical formula of CₙH₂ₙ; and n is a value between 3 and 6.

On fabricating the acidic IL, at first, a pyridine or a 1-butylimidazole is reacted with 1,3-propane sultone under 40° C. for 24 hours to obtain a white solid zwitterion. After being purified with ether and dried in vacuum, R⁺—(CH₂)₃—SO₃— is obtained, where R is pyridine or 1-butylimidazole. A certain amount of zwitterion is obtained to be added with a few moles of H₂SO₄ or R—SO₃H to be stirred under 80° C. for 4 hours for obtaining a viscous IL. Then, the impurities in IL are washed out with toluene and ether and dried in vacuum to obtain a [R⁺—(CH₂)₃—SO₃H][HSO₄⁻] or a [R⁺—(CH₂)₃—SO₃H][R—SO₃⁻]. The reaction equations are as follows:

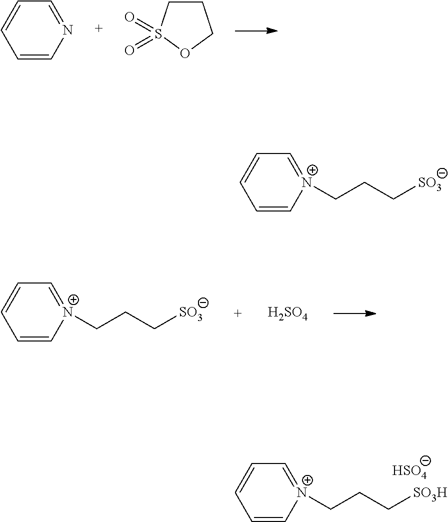

Then, a mixed solution of GMAE and HOAc is poured into the viscous IL for reaction by heating with 400 rpm of stirring. After the reaction, recycling HOAc and removing water through vacuuming, layers are formed by staying still and upper layer is taken out for quantitative analysis through gas chromatography (GC) to measure conversion rate of the GMAE. Therein, the reaction is processed at a temperature between 25° C. and 120° C. for a period of time between 0.1 and 5 hours with a mole ratio of HOAc/GMAE between 1.0 and 20 and a mole ratio of IL/GMAE between 0.02 and 1.0. For testing reuse of IL, the used IL is vacuumed under 95° C. to remove HOAc and water, and is separated from product as in step (c), and then a fresh feedstock solution is added for next acetylation.

### [State-of-Use 1] Various Temperatures

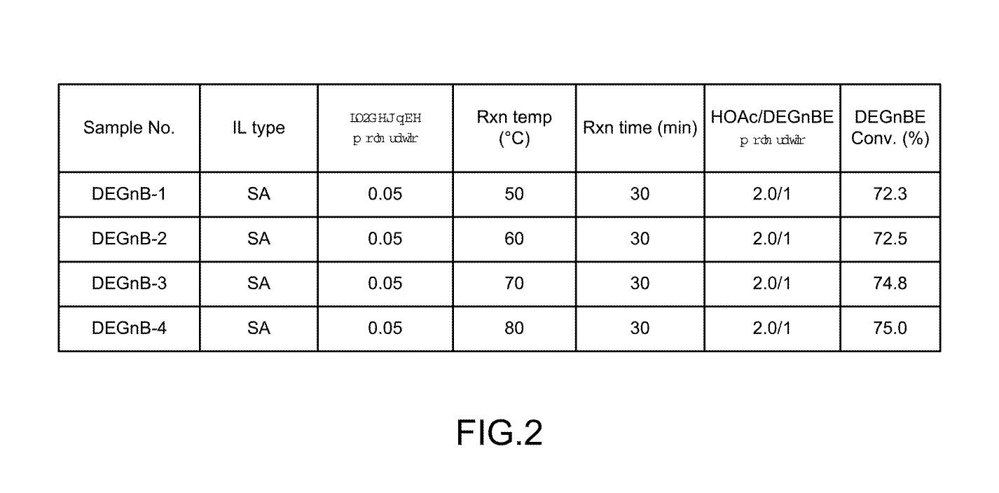

Please refer to FIG. 2, which is a view showing conversion rates for different temperatures. As shown in the figure, 1.01 g (0.005 mole) of PSPy solid is put in a bottle and 0.49 g (0.005 mole) of H₂SO₄ is gradually added with stirring under 60° C. for 30 minutes to obtain a viscous IL. Then a feedstock containing 16.2 g (0.1 mole) of DEGnBE and 12 g (0.2 mole) of HOAc are added to the IL for reaction with a stirring of 400 rpm for 2 hours at temperatures including 50° C., 60° C., 70° C. and 80° C. After reaction, it is vacuumed to remove HOAc and water and then is stayed still to obtain two layers—the upper layer is an ether-ester product and the lower layer is an IL. The product is taken out to be analyzed through GC for obtaining its composition (DEGnBE and DEGnBEA), acid value and conversion rate. As results show, increased conversion rates of DEGnBE acetylation are obtained with increased temperatures; however the increased amount is not much. Therein, under a 2.0 mole ratio of HOAc/DEGnBE and a 0.05 mole ratio of IL/DEGnBE, a preferred temperature for reaction is between 60° C. and 80° C.

### [State-of-Use 2] Various Reaction Periods of Time

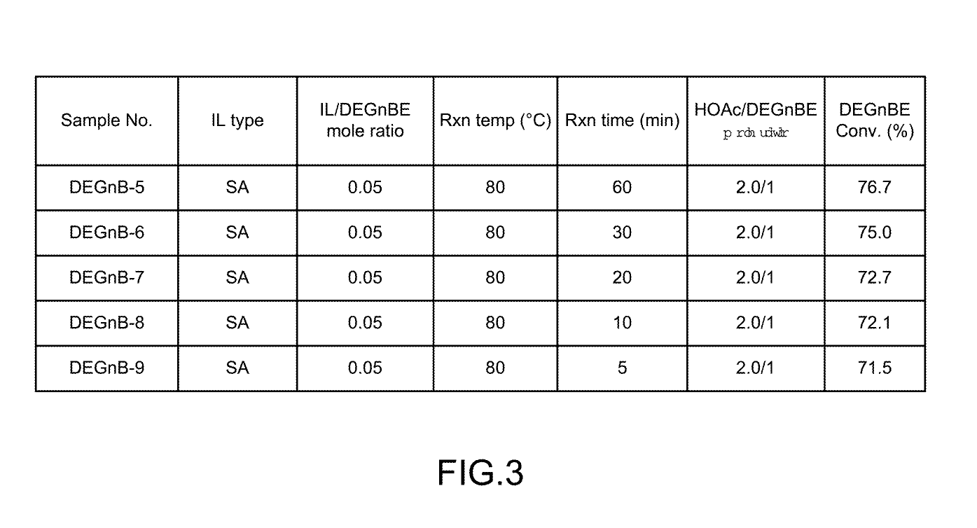

Please refer to FIG. 3, which is a view showing conversion rates for different reaction periods of time. As shown in the figure, through the same process used in State-of-use 1, the reactions are processed under 80° C. for 5, 10, 20, 30 and 60 minutes. As results show, with reaction period of time increased from 5 to 60 minutes, conversion rate is increased from 71.5% to 76.5%-20 to 60 minutes of reaction time is preferred.

### [State-of-Use 3] Various Mole Ratios

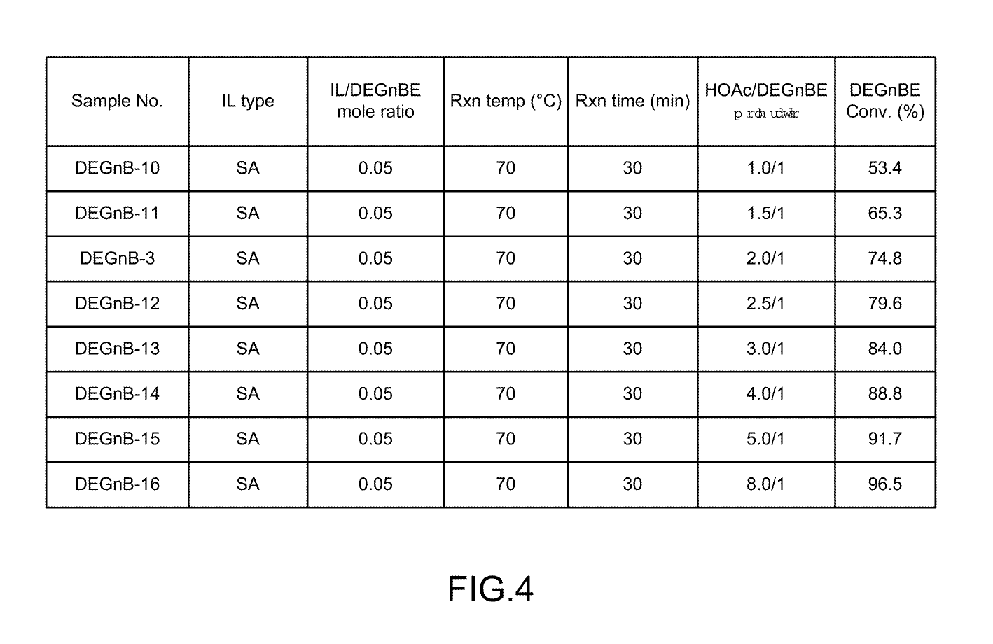

Please refer to FIG. 4, which is a view showing conversion rates for different mole ratios. As shown in the figure, through the same process used in State-of-use 1, the reaction is processed at 70° C. for 30 minutes with different mole ratios of HOAc/DEGnBE, which are 1.0/1, 1.5/1, 2.0/1, 2.5/1, 3.0/1, 4.0/1, 5.0/1 and 8.0/1. As results show, with increased mole ratios, DEGnBE conversion rates are increased from 53.4% for 1.0/1 mole ratio to 96.5% for 8.0/1 mole ratio. Since too much HOAc may result in problems of recycling HOAc, separating product from IL, a preferred mole ratio for HOAc/DEGnBE is between 2 and 5.

### [State-of-Use 4] Various IL Amounts

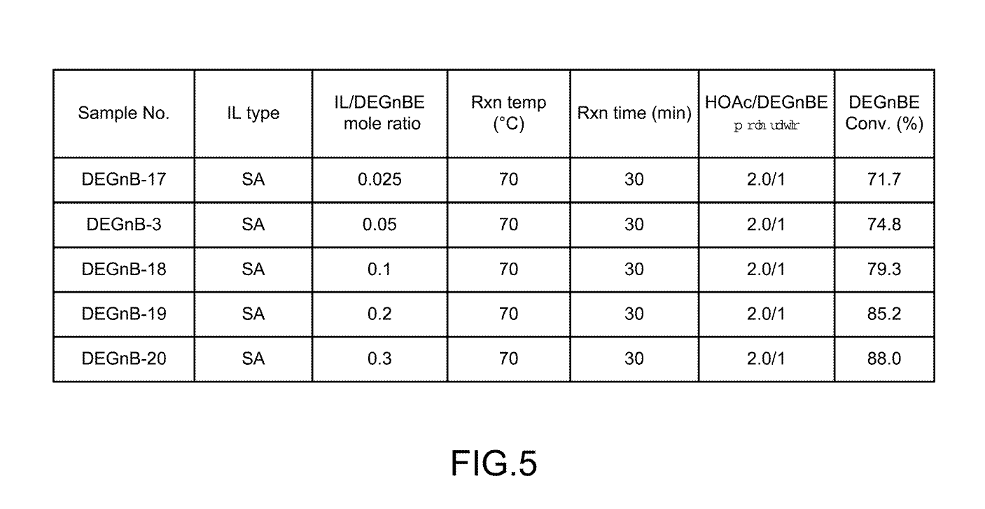

Please refer to FIG. 5, which is a view showing conversion rates for different amounts of IL. As shown in the figure, through the same process used in State-of-use 1, the reaction is processed under 70° C. for 30 minutes with different amount of IL, whose mole ratios of IL/DEGnBE are 0.025, 0.05, 0.1, 0.2 and 0.3. As results show, with the mole ratio of IL/DEGnBE increased from 0.025 to 0.3, the conversion rate is increased from 71.7% to 88.0%. Since too high mole ratio may result in problems of recycling IL, a preferred mole ratio is between 0.05 and 0.1.

### [State-of-Use 5] Various Acids

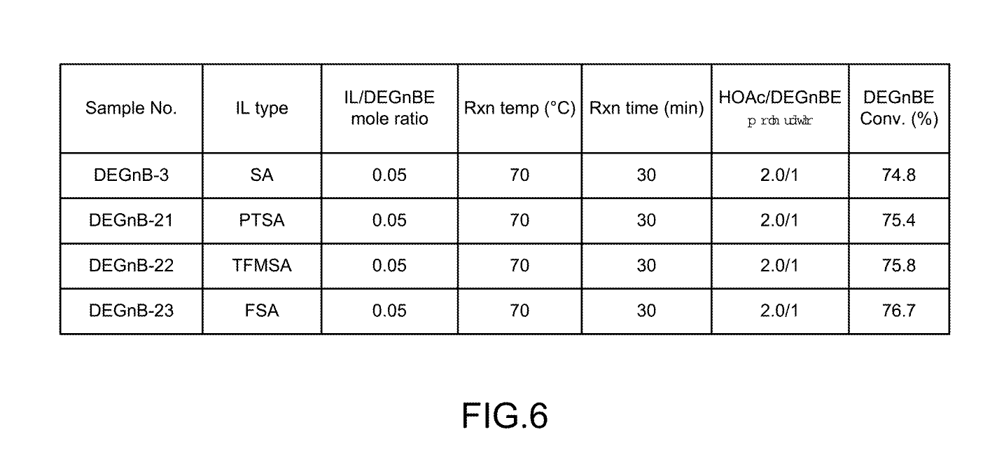

Please refer to FIG. 6, which is a view showing conversion rates for different ILs. As shown in the figure, through the same process used in State-of-use 1, the reaction is processed under 70° C. for 30 minutes at a 2.0 mole ratio of HOAc/DEGnBE with various acidic IL. The acidic IL is fabricated by a zwitterion (PSPy) with SA, FSA, TFMSA or P-TSA. PSPy can be added with methanol before being mixed with a strong acid for forming an IL by stirring; and then the methanol can be removed by vacuuming. As results show, conversion rates for the four acids are similar, while SA and P-TSA are preferred for easy handling.

### [State-of-Use 6] ILs Containing Various Water Amounts

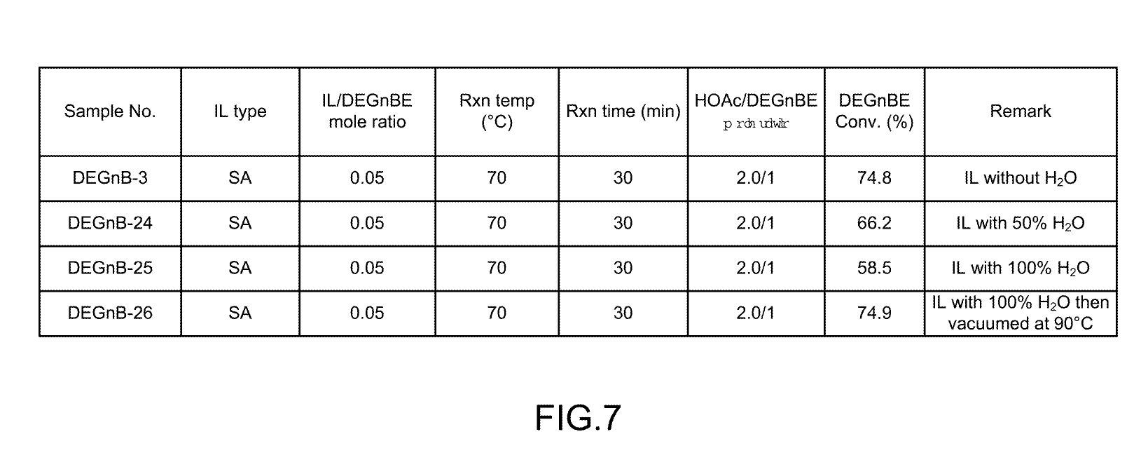

Please refer to FIG. 7, which is a view showing conversion rates for ILs containing different amounts of water. As shown in the figure, through the same process used in State-of-use 1, the reaction is processed at 70° C. for 30 minutes with 2.0 mole ratio of HOAc/DEGnBE. Before the reactions, 50 wt % and 100 wt % of water are added to the IL respectively and are stirred for 20 minutes. As results show, conversion rates are lowered down from 74.8% (with no water) to 66.2% (with 50 wt % water) and 58.5% (with 100 wt % water). It proves that water can weaken the reaction. For another test, 100 wt % of water is added into the IL and then is removed by vacuuming at 90° C., followed by the acetylation reaction. The reaction result is similar to that catalyzed by IL without adding water. It proves that the water generated from the reaction could be removed by vacuuming and the vacuumed IL has similar catalytic activity as fresh IL.

### [State-of-Use 7] Various Water-Carrying Agent

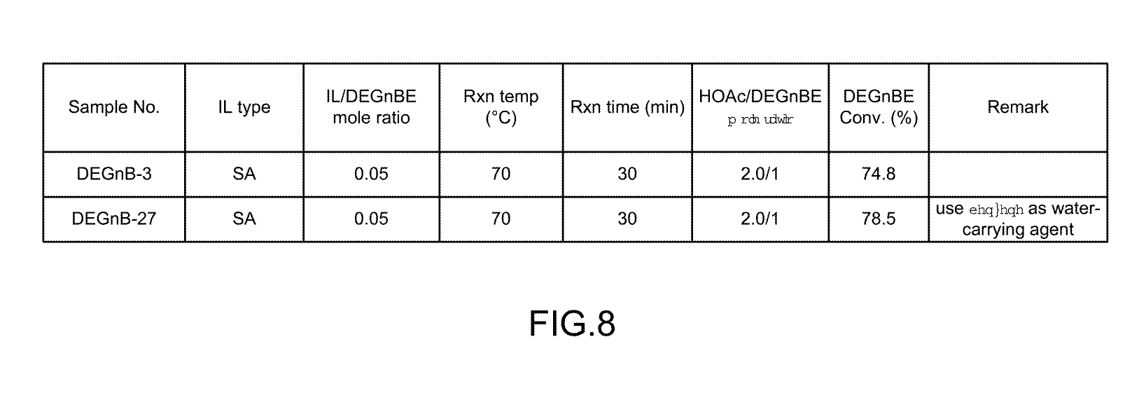

Please refer to FIG. 8, which is a view showing the conversion rates with the water-carrying agent used. As shown in the figure, through the same process used in State-of-use 1, DEGnBE and HOAc are reacted to obtain DEGnBEA and the same mole of water. With the same mole of benzene added, an water-benzene azeotrope (whose boiling point is 69.3° C.) is formed. Thus, the generated water can be removed. As results show, conversion rates are increased from 74.8% to 78.5%.

### [State-of-Use 8] Various Alkyl Ethers

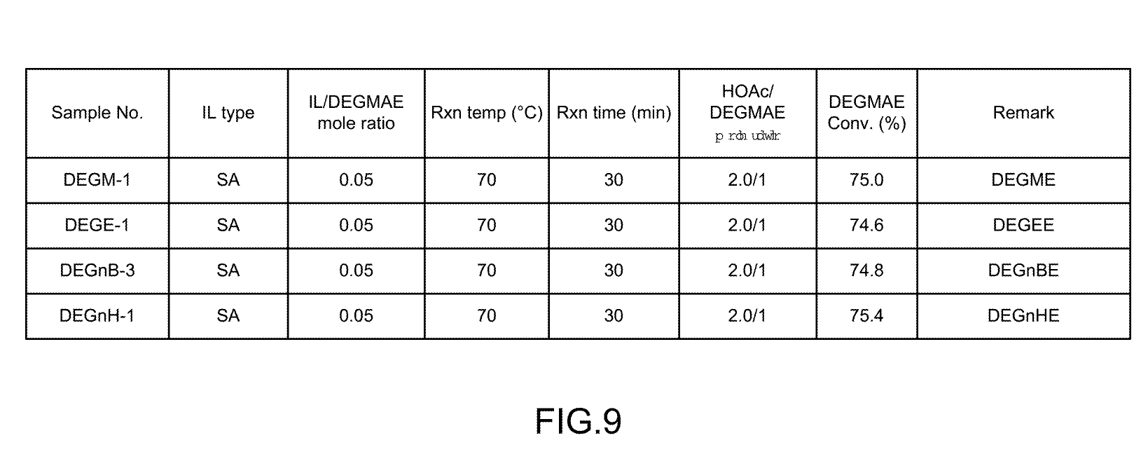

Please refer to FIG. 9, which is a view showing the conversion rates for different DEGMAEs. As shown in the figure, through the same process used in State-of-use 1, an IL of [PSPy][H₂SO₄] is used as a catalyst, where IL/DEGMAE mole ratio is 0.05; the GMAE is DEGME, DEGEE, DEGnBE or DEGnHE; HOAc/DEGMAE mole ratio is 2.0; the reaction is proceeded at 70° C. for 30 minutes by stirring. After the reaction, product is obtained by staying still, where selectivity of the products are as follows: DEGME>DEGEE>DEGnBE>DEGnHE.

### [State-of-Use 9] Various Glycol N-Butyl Ethers

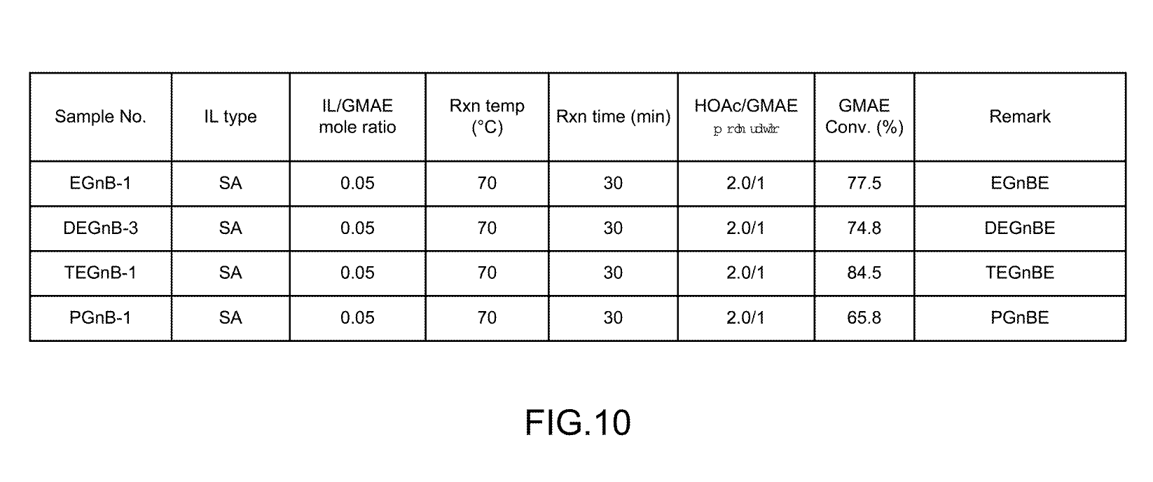

Please refer to FIG. 10, which is a view showing the conversion rates for different glycol n-butyl ethers. As shown in the figure, through the same process used in State-of-use 1, an IL of [PSPy][H₂SO₄] is used as a catalyst, where an IL/GMAE mole ratio is 0.05; a GMAE is EGnBE, DEGnBE, TEGnBE or PGnBE; an HOAc/GMAE mole ratio is 2.0; and the reaction is proceeded at 70° C. for 30 minutes by stirring. As results show, all kinds of the GMAE obtains high conversion rates and products are leveled out by staying still. Selectivities of the products are as follows: DEGnBE>PGnBE>TEGnBE>EGnBE, where the selectivity can be improved through changing reaction conditions.

### [State-of-Use 10] Reused IL

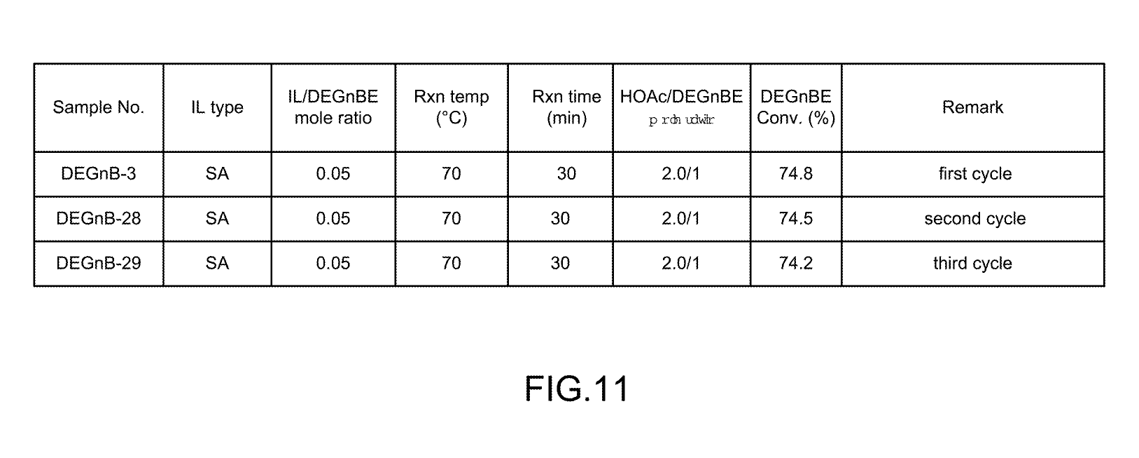

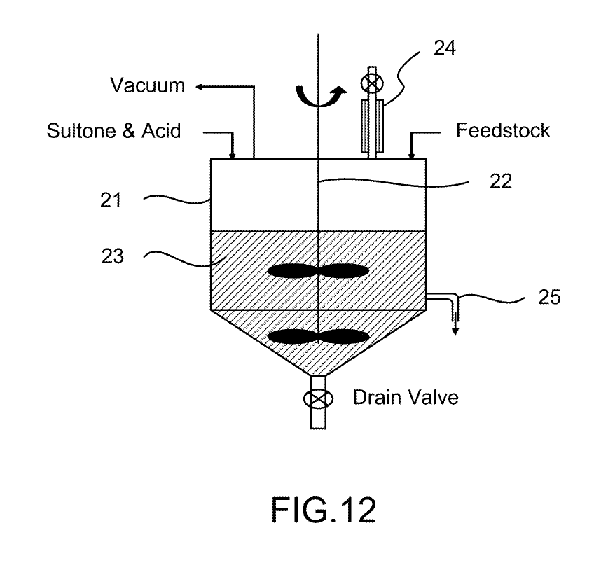

Please refer to FIG. 11 and FIG. 12, which are a view showing conversion rates for different reused acidic IL; and a view showing equipments of the preferred embodiment. As shown in the figures, through the same process used in State-of-use 1, the reaction is processed under 70° C. for 30 minutes with an IL of [PSPy][SA] as a catalyst, where mole ratios of IL/DEGnBE and HOAc/DEGnBE are 0.05 and 2.0 respectively. After HOAc and water are removed through vacuuming under 95° C., two layers are formed by staying still. The upper layer of product is taken out and the lower layer of IL is recycled for next reaction. As results show, after being recycled for two times, the conversion rates remain high. Equipments designed for the present disclosure is described in FIG. 12. At first, the zwitterion and the strong acid is added into a reactor 21 to be mixed by stirring with a stirring part 22 at a temperature between 70° C. and 80° C. for obtaining a viscous IL 23. Then, a mixed solution of HOAc and DEGnBE is added to be stirred under a temperature between 70° C. and 80° C. for 30 minutes. And, then, the reactor 21 is vacuumed for recycling unreacted HOAc from a condenser 24 and removing the generated water. Then, it is stayed still for forming two layers. The upper layer of product is taken out from a reactor side pipe 25 and the lower layer of IL is reused for next reaction.

To sum up, the present disclosure is a method of fabricating GMAEA using acidic IL catalyst, where a Bronsted acidic IL is used as a catalyst for acetylation of GE and a product is obtained with the acidic IL recyclable for reuse.

The preferred embodiment herein disclosed is not intended to unnecessarily limit the scope of the disclosure. Therefore, simple modifications or variations belonging to the equivalent of the scope of the claims and the instructions disclosed herein for a patent are all within the scope of the present disclosure.

What is claimed is:

1. A method of fabricating glycol monoalkyl ether acetate (GMAEA) using acidic ionic liquid (IL) catalyst, comprising the steps of:
(a) reacting an organic nitride compound with alkyl sultone to obtain a zwitterion; and, after purifying and drying said zwitterion, processing a reaction with a strong acid (SA) having a sulfuric group (—SO₄H) or a sulfonic group (—SO₃H) to obtain a viscous water-tolerable Bronsted acidic IL,
wherein said SA has a group selected from a group consisting of a sulfuric group (—SO4H) and a sulfonic group (—SO3H); and
wherein a mole ratio of said strong acid to said zwitterion (SA/Zw) is between 1.0 and 1.5;
(b) adding a hot solution of GMAE and acetic acid (HOAc) into said IL to process an acetylation of said GMAE,
wherein a mole ratio of said IL to said GMAE (IL/GMAE) is between 0.02 and 1.0;
wherein a mole ratio of HOAc to said GMAE (HOAc/GMAE) is between 1.0 and 20; and
wherein said acetylation is processed at a temperature between 25° C. and 120° C. for a period between 0.1 and 5 hours; and
(c) after removing CH₃OH and H₂O under vacuum, staying still at a high temperature to obtain two layers of materials and obtaining a product being a lighter layer of said layers with said IL being a heavier layer of said layers.

2. The method according to claim 1,
wherein, in step (b), a water-carrying agent is added to improve a conversion rate of said acetylation.

3. The method according to claim 2,
wherein said water-carrying agent is selected from a group consisting of benzene and toluene.

4. The method according to claim 1,
wherein said GMAE is an ethylene glycol monoalkyl ether having a chemical formula of HO—[C₂H₄O]ₘ—R; and
wherein m is a value between 1 and 3, R is CₙH₂ₙ₊₁) and n is a value between 1 and 9.

5. The method according to claim 1,
wherein said GMAE is an propylene glycol monoalkyl ether having a chemical formula of HO—[CH₂CH(CH₃)O]ₘ—R; and
wherein m is a value between 1 and 3, R is CₙH₂ₙ₊₁) and n is a value between 1 and 9.

6. The method according to claim 1,
wherein said organic nitride compound is a nitrogen-containing compound having an element selected from a group consisting of imidazole, pyridine, and alkyl amine; and
wherein said organic nitride compound is reacted with alkyl sultone to obtain said zwitterion as a precursor of said acidic IL.

7. The method according to claim 6,
wherein alkyl in said organic nitride compound is a compound selected from a group consisting of alkylimidazole, alkylpyridine and alkyl amine has a chemical formula of CₙH₂ₙ₊₁) and n is a value between 1 and 18.

8. The method according to claim 6,
wherein alkyl in said alkyl sultone has a chemical formula of CₙH₂ₙ, and n is a value between 3 and 6.

9. The method according to claim 1,
wherein said strong acid is selected from a group consisting of sulfuric acid (H₂SO₄, SA) and alkyl sulfonic acid (R—SO₃H).

10. The method according to claim 9,
wherein said R—SO₃H is selected from a group consisting of fluorosulfonic acid (FSO₃H, FSA), trifluoro methane sulfonic acid (CF₃SO₃H, TFMSA) and p-toluene sulfonic acid (p-CH₃—C₆H₄—SO₃H, P-TSA).

11. The method according to claim 1,
wherein said mole ratio of SA/Zw has a preferred value between 1.0 and 1.2.

12. The method according to claim 1,
wherein said mole ratio of IL/G MAE has a preferred value between 0.05 and 0.1.

13. The method according to claim 1,
wherein said mole ratio of HOAc/GMAE has a preferred value between 2 and 5.

14. The method according to claim 1,
wherein said acetylation is processed at a preferred temperature between 60 and 80 Celsius degrees.

15. The method according to claim 1,
wherein said acetylation is processed for a preferred period of time between 20 and 60 minutes.
